SEQUENCE LISTING (5 sequences)

SEQ ID NO: 1 - protein, 13 aa, Homo sapiens
QEREGSSLDSPRS

SEQ ID NO: 2 - protein, 7 aa, Homo sapiens
SLDSPRS

SEQ ID NO: 3 - DNA, 20 bp, Artificial Sequence
  Synthetic Construct
cgctgcccagatcaagagtc

SEQ ID NO: 4 - DNA, 22 bp, Artificial Sequence
  Synthetic Construct
agatgctgacaaccaggaaagg

SEQ ID NO: 5 - protein, 57 aa, Homo sapiens
APFRSALESSPADPATLSEDEARLLLAALVQDYVQNKASELEQEQEREGSSLDSPRS

(19) United States
(12) Patent Application Publication
(10) Pub. No.: US 2013/0046085 A1
(43) Pub. Date: Feb. 21, 2013

(54) ANTIBODIES AGAINST N-PROCALCITONIN

(71) Applicants: Eva TAVARES VÁZQUEZ, Sevilla (ES); Javier MIÑANO SÁNCHEZ, Sevilla (ES)

(72) Inventors: Eva TAVARES VÁZQUEZ, Sevilla (ES); Javier MIÑANO SÁNCHEZ, Sevilla (ES)

(73) Assignees: Universidad De Sevilla, Sevilla (ES); Fundacion Publica Andaluza Para La Gestion De La Investigacion De La Salud En S, Sevilla (ES)

(21) Appl. No.: 13/527,069

(22) Filed: Jun. 19, 2012

Related U.S. Application Data
(60) Provisional application No. 61/499,808, filed on Jun. 22, 2011.

(51) Int. Cl. C07H 21/04

(52) U.S. Cl. 536/23.53

(57) ABSTRACT

Specific antibodies against N-procalcitonin, peptides, genetic constructions and methods for the obtainment of the peptides used in the obtainment of the antibodies. These antibodies can be used for preparing drugs, or diagnostic kits for diseases that develop with systemic inflammatory response or metabolic stress.

### CROSS-REFERENCE TO RELATED APPLICATIONS

This application claims priority under 35 U.S.C. §119(e) to U.S. Provisional Patent Application No. 61/499,808, filed Jun. 22, 2012 and entitled “Antibodies Against N-Procalcitonin” in the name of Eva TAVARES VÁZQUEZ et al., which is incorporated by reference herein in its entirety.

### FIELD

The present invention is within the field of biotechnology and medicine. It relates to antibodies against N-procalcitonin, peptides, genetic constructions and the methods for the obtainment of the peptides used in the obtainment of the antibodies, and the uses of said antibodies.

### PRIOR ART

Procalcitonin (PCT or proCT) is a glycopeptide hormone of 116 amino acids and approximately 13 kDa of molecular weight. This molecule is the precursor of calcitonin. Its synthesis starts with the transcription of the Calca-1 gene situated in chromosome 11p. This subscript is subsequently processed giving rise to preprocalcitonin, precursor of PCT. This precursor is composed of 141 amino acids and its later processing gives rise to PCT. Its amino acid sequence was already described in 1984 (Moullec et al. 1984. FEBS lett. 167: 93-97)

This PCT, for its part, suffers successive digestions to give rise to three different molecules: aminoprocalcitonin (N-procalcitonin, N-PCT or N-proCT) composed of 57 amino acids of the N-terminal zone; calcitonin in immature and inactive form, formed by 33 amino acids of the central zone of the PCT; and peptide corresponding to the C-terminal zone, formed by 21 amino acids (residues 96-116 of PCT) and called CCP-I or katacalcin (Jacobs et al. 1981. J Biol Chem. 256:1803-2807; Steenbergh et al. 1986. FEBS lett. 209: 97-103). In normal physiological conditions (not pathological), these molecules are produced as a result of a proteolytic intracellular process carried out by the prohormone enzyme conversase in the C-cells of the thyroids and in the neuroendocrine cells of the lung.

Except in the case of calcitonin (CT), the physiological effects of all these peptides are not well known. Despite this, it has been observed that there is an important increase in the circulating and brain levels of both PCT and N-PCT in situations of inflammation, infection and sepsis (Whang et al. 1998. J Clin Endocrinol Metab. 83: 3296-3301). Due to the increase in the two molecules, a better structural knowledge of the PCT against N-PCT, and the existence of commercial kits, to date the use has been suggested of antibodies against PCT both as diagnostic marker (U.S. Pat. No. 6,451,311 B2) and for the therapy of sepsis and systemic inflammatory response syndrome (SIRS) (WO 98/33524).

Despite this, the physiological role of PCT as well as its systemic effects are not well known. Their involvement is, however, known in systemic inflammatory response due to its relation with various cytokines and its increase as a response to bacterial toxins (Brunkhorst et al. 1998. Intensive Care Med. 24: 888-889). Despite this, recent studies indicate that PCT in itself has a low or zero biological activity, in addition to the existence of contradictory studies on its effects in various in vitro models. These results do not justify its supposed role as secondary mediator in sepsis.

For its part, N-procalcitonin, unlike PCT, katacalcin or CT, has demonstrated that it is a highly conserved peptide with a structural homology over 90% in all mammal species studied, which suggests an important role on a biological level. This protein also has a marked biological activity in hypermetabolism situations such as obesity, fasting, etc. i.e. situations of metabolic stress. Through different studies, it was observed that in normal conditions N-PCT is expressed in brain regions involved in the control of energy homeostasis (Ojeda et al. 2006. Neurosci Lett. 408: 40-45; Tavares et al. 2007. Endocrinology. 148: 1891-1901). It is also observed that N-PCT is increased in the case of administration of bacterial endotoxin (Tavares et al. 2005. Clin Diagn Lab Immunol. 12: 1085-1093) suggesting a role in the inflammatory response. Furthermore, it has been demonstrated that the central administration of N-PCT simulates the inflammatory responses that occur in sepsis (lethargy, fever, anorexia, weight reduction), indicating its importance in the inflammatory response via mechanisms dependent on the activation of POMC neurons and prostaglandin synthesis. Therefore, it is a protein that has awoken great interest as secondary mediation in the systemic inflammatory response syndrome. Furthermore, it may be useful as diagnostic marker in sepsis (Jones et al. Ann emerg med. 2007. 50: 47-51).

At present, a combination of antikatacalcin and anti-calcitonin antibodies is being used to detect calcitonin precursor molecules in sepsis (EP 0656121 B1). This detection system is very non-specific and may also require the use of two antibodies. It also has the disadvantage that it does not provide any information on the physiological role of PCT. Therefore, it is necessary to have a more specific and relevant marker that provides greater sensitivity to the sepsis detection systems and therefore allows a greater and faster diagnosis. Furthermore, it is necessary that this marker makes it possible to better decipher the pathogenic mechanisms involved in this syndrome. In this respect, N-PCT is postulated as a highly important marker due to its involvement in the processes of sepsis or SIRS. Furthermore, its blocking could produce the inhibition of some of the processes in which it is involved, for example, as fever or anorexia in sepsis, with the consequent improvement of patients. Despite this, at present, the detection systems are very non-specific, since they do not allow the exclusive recognition of this molecule, but they largely have cross-reactions with procalcitonin (Whang et al. 1998. J Clin Endocrinol Metab. 83: 3296-3301).

Therefore, it is necessary to search for elements that allow the detection or blocking of N-PCT, the molecule involved in diseases with alteration of the systemic inflammatory response. One of the elements that are postulated as of greatest importance in this respect would be antibodies highly specific against this peptide.

### DESCRIPTION OF THE INVENTION

There is, therefore, the need to find a tool that allows the detection of N-procalcitonin as well as its blocking without having effects on other molecules. The authors of the present invention disclose a method for obtaining antibodies capable of detecting or blocking N-procalcitonin, the peptides used in the production of said antibodies, and a method for the detection of diseases that develop with alteration of the systemic inflammatory response or with metabolic stress.

In this sense, a first aspect of the present invention relates to an isolated nucleotide sequence, hereinafter first nucleotide sequence of the invention, which codes for the sequence SEQ ID NO:1, or for a biologically active fragment or variant of SEQ ID NO: 1. SEQ ID NO:1 corresponds to the amino acid sequence of the last 13 residues of the protein N-PCT.

The term “isolated”, as used in this specification, refers to nucleotides or peptides which: 1) are substantially free from components that normally accompany or interact with it in nature, or 2) if it is found in its natural medium, they have been synthetically (not naturally) altered by human intervention and/or introduced in a cell which does not possess it. For example, a natural nucleotide becomes “isolated” if it has been altered, or comes from DNA which has been altered by human intervention (by means of, for example, but without limiting ourselves to, directed mutagenesis, insertions, deletions, etc). Likewise, a natural nucleotide becomes “isolated” if it is introduced by non-natural means in a non-native genome to said nucleotide (transfection). Therefore, the term “isolated” in this last case, is equivalent to the term “heterologous”.

In the present invention, variant or biologically active fragment is understood to be those variants or fragments of the peptides indicated that have an identical physiological, metabolic or immunological effect, or have the same use as those described. In other words, they are functionally equivalent. Said effects can be determined by conventional methods such as those described in the examples accompanying this description. Particularly, the term “variant” relates to a peptide substantially homologous to any of the peptides whose amino acid sequence is set down in SEQ ID NO: 1 or SEQ ID NO: 2. In general, a variant includes additions, deletions or substitutions of amino acids. The term “variant” also includes the peptides resulting from post-translational modifications such as, but without being limited to, glycosylation, phosphorylation or methylation.

As used here, a peptide is “substantially homologous” to any of SEQ ID NO: 1 or SEQ ID NO: 2 peptides, when their sequence of amino acids has a good alignment with the sequence of amino acids SEQ ID NO: 1 or SEQ ID NO: 2, respectively; i.e. when their sequence of amino acids has a degree of identity with respect to the sequence of amino acids SEQ ID NO: 1 or SEQ ID NO: 2, respectively of, at least, 50%, typically of, at least, 80%, advantageously of, at least, 85%, preferably of, at least 90%, more preferably of, at least, 95%, and, even more preferably of, at least, 99%. The sequences homologous to any of SEQ ID NO: 1 or SEQ ID NO: 2 peptides can be easily identified by a person skilled in the art, for example, with software suitable for comparing sequences.

The term “identity”, as used in this specification, makes reference to the proportion of nucleotides or identical amino acids between two nucleotide or amino acid sequences compared. The methods of comparison of sequences are known in the state of the art and include, although without being limited to, the GAG program, including GAP (Devereux et al., Nucleic Acids Research 12: 287 (1984) Genetics Computer Group University of Wisconsin, Madison, (WI); BLAST, BLASTP or BLASTN, and FASTA (Altschul et al., J. Mol. Biol. 215: 403-410 (1999).

A second aspect of the present invention relates to an isolated nucleotide sequence, hereinafter second nucleotide sequence of the invention, which codes for the sequence SEQ ID NO:2, or for a biologically active fragment or variant of SEQ ID NO: 2. SEQ ID NO:2 corresponds to the amino acid sequence corresponding to the last 7 residues of the protein N-PCT.

A third aspect of the present invention relates to a peptide, hereinafter first peptide of the invention, that consists of the amino acid sequence SEQ ID NO:1, or in a biologically active fragment or variant of SEQ ID NO: 1.

A fourth aspect of the present invention relates to a peptide, hereinafter second peptide of the invention, which consists of the amino acid sequence SEQ ID NO:2, in a biologically active fragment or variant.

Another aspect of the present invention relates to a genetic construction, hereinafter genetic construction of the invention, comprising: a) nucleotide sequence comprising a sequence that codes for SEQ 30 ID NO:1 or SEQ ID NO:2, its variants or biologically active fragments, or b) nucleotide sequence comprising a sequence that codes for SEQ ID NO:1 or SEQ ID NO:2, its variants or biologically active fragments, included in an expression vector, operationally bound to, at least, one promoter which directs the transcription of said sequence of nucleotides of interest, and with other sequences necessary or appropriate for their suitable transcription and regulation in time and place, for example initiation and termination signals, cleavage sites, polyadenylation signals, replication origin, transcriptional enhancers, transcriptional silencers, etc . . . . Multiples of these systems or expression vectors can be obtained by conventional methods known by persons skilled in the art (Sambrook et al., 1989) and form part of the present invention.

Another aspect of the present invention relates to a host cell, hereinafter host cell of the invention, comprising any of the nucleotide sequences of the invention, or the genetic construction of the invention.

A “host” or “host cell” as is used in this specification relates to a cell that serves as recipient of the genetic construction and serves as vehicle for its expression. A host cell or host may also indicate a cell or host that expresses a protein or a recombinant peptide of interest where the host cell is transformed with an expression vector containing the gene of interest. In a preferred embodiment, the host cell is a prokaryotic cell. In another preferred embodiment, the host cell is a eukaryotic cell.

Another aspect of the present invention relates to the use of a nucleotide sequence of the invention, of the genetic construction of the invention, or of the host cell of the invention, for the generation of recombinant peptides. Said recombinant peptides that will comprise the amino acid sequence is that of any of the peptides of the invention, or of any of their variants or biologically active fragments.

Another aspect of the present invention relates to the use of the peptides of sequence SEQ ID NO: 1 or SEQ ID NO:2, their variants or biologically active fragments, or recombinant peptides, for the generation of antibodies.

Another aspect of the present invention relates to a method for the generation of antibodies (hereinafter first method of the invention) comprising the following steps:
c) adding a cysteine at one of the ends of a peptide of the invention,
d) conjugating the peptide with KLH (Keyhole Limpet Hemocyanin),
e) immunizing a mammal animal with a peptide according to (c),
f) extracting the antiserum from the animal, and
g) purifying the antibody that specifically recognizes N-procalcitonin.

Additionally, the first method of the invention may include a previous step, which consists of the generation of the peptide(s) of the invention, of step (c), in recombinant manner, by the aforementioned procedure. In a preferred embodiment of this aspect of the invention the mammal host animal is a rodent. In a more preferred embodiment the rodent is a mouse. In another preferred embodiment the mammal host animal is a rabbit.

The addition of the cysteine to the peptides of the invention causes an increase in stability of the molecule as well as the improvement of their pharmacokinetic characteristics, allowing a better response to immunization of the animals.

In this specification, “animal” is understood to be any organism of the superkingdom Eukaryota and kingdom Metazoa. The term “mammal” is used to refer to any organism of the superkingdom Eukaryota, kingdom Metazoa, phylum Chordata, subphylum Craniata, superclass Gnathostomata and class Mammalia.

Another aspect of the present invention relates to a method for the generation of antibodies (hereinafter second method of the invention) comprising the following steps:
h) adding a cysteine at one of the ends of a peptide of the invention
i) conjugating it with KLH,
j) immunizing a mammal animal with a peptide according to (g),
k) analysing the titration against the peptide of step (g) with ELISA, in the mammal animal of step (h),
l) fusing the splenocytes of the host animals for the generation of immortalized cell lines,
m) expanding the clones,
n) selecting the best producers.

Additionally, the first method of the invention may include a previous step, which consists of the generation of the peptide or peptides of the invention, of step (h), in recombinant manner, by the previously described procedure. In a preferred embodiment of this aspect of the invention the mammal host animal is a rodent. In a more preferred embodiment the rodent is a mouse. In another preferred embodiment the mammal host animal is a rabbit.

Another aspect of the present invention relates to the antibodies generated by the first or second method of the invention. In a preferred embodiment of this aspect of the invention, the antibodies specifically recognize N-procalcitonin. In a more preferred embodiment the antibodies are capable of binding to the amino acid sequences SEQ ID NO:1 and/or SEQ ID NO:2.

Another aspect of the present invention relates to the use of the antibodies of the present invention for the preparation of a drug for the treatment of diseases that develop with alterations of the inflammatory response. In a preferred embodiment of this aspect of the invention the disease that develops with alteration of the inflammatory response is selected from the list comprising: sepsis, septic shock, cardiogenic shock, post-surgical complications, peritonitis, transplants, autoimmune diseases, obesity, diabetes, bacterial meningitis, neoplasias or neurodegenerative diseases that develop with inflammation.

In the present invention, alteration in inflammatory response is understood by the clinical response characterized by tachycardia, tachypnea, fever or hypothermia, and leukopenia or leukocytosis. It may be caused by a serious bacterial infection sepsis, a severe trauma or a serious pancreatitis.

Another aspect of the present invention relates to the use of the antibodies of the present invention for the preparation of a drug for the treatment of diseases that develop with metabolic stress.

In the present invention, metabolic stress is understood to be the response developed by the organism to any type of aggression, which is characterized. Its magnitude depends on the type and intensity of the aggression and evolves over time. It is characterized in that there is an increase in energy expenditure and the consumption of oxygen, hypotension, induced by the increase in the circulating levels of certain hormones (glucocorticoids, ACTH, . . . ), cytokines, lipid mediators, etc., and their magnitude evolves over time, it depends on the type and intensity of the aggression and their magnitude evolves over time. There are multiple diseases that present this, for example, although without limiting ourselves to, diabetes, obesity, severe trauma, serious infectious processes or septic shock.

The term “drug”, as used in this specification, makes reference to any substance used to prevent, diagnose, relieve, treat or cure diseases in man and in animals. Within the context of the present invention it relates to a preparation comprising the antibody or antibodies of the invention.

As used here, the term “active principle”, “active substance” “pharmaceutically active substance”, “active ingredient” or “pharmaceutically active ingredient” means any component that potentially provides a pharmacological activity or another different effect in the diagnosis, cure, mitigation, treatment or prevention of a disease or which affects the structure or function of the body of man or other animals. The term includes those components that promote a chemical change in the preparation of the drug and are present therein in a modified form provided that it provides the specific activity or the effect.

Another aspect of the present invention relates to the use of the antibodies of the present invention for the quantification of N-procalcitonin in serum, cerebrospinal fluid, cell or tissue homogenates, or other biological fluids.

Another aspect of the present invention relates to the use of the antibodies of the present invention for the study or the early diagnosis of diseases that develop with alterations of the inflammatory response. In a preferred embodiment of this aspect of the invention the disease that develops with alteration of the inflammatory response is selected from the list comprising: sepsis, septic shock, cardiogenic shock, post-surgical complications, peritonitis, transplants, autoimmune diseases, obesity, diabetes, bacterial meningitis, neoplasias or neurodegenerative diseases that develop with inflammation.

Another aspect of the present invention relates to the use of the antibodies of the present invention for the study or the early diagnosis of diseases that develop with metabolic stress.

Another aspect of the invention relates to a method of obtainment of data useful for the diagnosis of diseases that develop with alterations of the inflammatory response, hereinafter third method of the invention, comprising:
o) obtaining an isolated biological sample from an individual,
p) detecting the quantity of N-procalcitonin in the biological sample of (o), by the antibodies of the invention, and
q) comparing the quantities obtained in step (p) with a reference quantity.

Another aspect of the invention relates to a diagnostic method of diseases that develop with alterations of the inflammatory response, of aorta hereinafter fourth method of the invention, comprising steps (o)-(p) of the third method of the invention, and furthermore:
r) assigning an individual according to step (o) to the group of individuals with disease that develops with alteration of the inflammatory response when a quantity of N-procalcitonin is detected in step (p) greater and statistically significant in comparison with a reference quantity.

Steps (p) and/or (q) of the methods described above may be totally or partially automated, for example, by a robotic sensor apparatus to detect the quantity in step (p) or computerized comparison in step (q).

In a preferred embodiment of this aspect of the invention, the disease that develops with alteration of the inflammatory response is selected from the list comprising: sepsis, septic shock, cardiogenic shock, post-surgical complications, peritonitis, transplants, autoimmune diseases, obesity, diabetes, bacterial meningitis, neoplasias or neurodegenerative diseases that develop with inflammation.

Another aspect of the invention relates to a method of obtainment of data useful for the diagnosis of diseases that develop with alterations of the metabolic stress, comprising:
s) obtaining an isolated biological sample from an individual,
t) detecting the quantity of N-procalcitonin in the biological sample of (q), by the antibodies of the invention, and
u) comparing the quantities obtained in step (t) with a reference quantity.

Another aspect of the invention relates to a diagnostic method of diseases that develop with alterations of the inflammatory response, hereinafter fourth method of the invention, comprising steps (s)-(t) of the third method of the invention, and furthermore:
v) assigning an individual according to step (s) to the group of individuals with disease that develops with alteration of the inflammatory response when quantity of N-procalcitonin is detected in step (t) greater and statistically significant in comparison with a reference quantity.

Steps (t) and/or (u) of the methods described above may be totally or partially automated, for example, by a robotic sensor apparatus to detect the quantity in step (t) or computerized comparison in step (u).

The term “diagnostic”, as used in the present invention, relates to the capacity of discriminating between individuals affected by diseases that develop with alterations of the inflammatory response. It also relates, but without limiting ourselves, to the capacity of discriminating between samples from patients that have different stages of said diseases. This discrimination as understood by a person skilled in the art cannot aim to be correct in 100% of the samples analysed. However, it requires that a statistically significant quantity of the samples analysed are correctly classified. The quantity that is statistically significant can be established by a person skilled in the art by the use of different statistical tools, for example, but without being limited to, by the determination of confidence intervals, determination of p value, Student test or Fisher's discriminating functions. Preferably, the confidence intervals are at least 90%, at least 95%, at least 97%, at least 98% or at least 99%. Preferably, the p value is less than 0.1, 0.05, 0.01, 0.005 or 0.0001. Preferably, the present invention makes it possible to correctly detect the disease in differential manner in at least 60%, at least 70%, at least 80%, or at least 90% of the subjects of certain group of population analysed.

An “isolated biological sample” includes, but is not limited to, cells, tissues and/or biological fluids of an organism, obtained by any method known by a person skilled in the art. Preferably, the isolated biological sample is a biological fluid, such as, for example, but without being limited to, blood, plasma or blood serum. More preferably, the biological fluid is blood serum or cerebrospinal fluid.

The term “individual”, as used in the description, relates to animals, preferably mammals, and more preferably, humans. The term “individual” does not aim to be limiting in any aspect, and can be of any age, sex and physical condition.

Another aspect of the invention relates to a diagnostic kit of diseases that develop with alterations of the inflammatory response comprising the antibodies of the invention. Said kit may additionally contain, but without any type of limitation, buffers, agents to prevent contamination, protein degradation inhibitors, etc. On the other hand, the kit may include all the supports and receptacles necessary to implement and optimize the third and fourth method of the invention.

Another aspect of the invention relates to a diagnostic kit of diseases that develop with metabolic stress, comprising the antibodies of the invention.

Throughout the description and the claims the word “comprises” and its variants are not intended to exclude other technical characteristics, additives, components or steps. For persons skilled in the art, other objects, advantages and characteristics of the invention will be inferred in part from the description and in part from the practice of the invention. The following figures and examples are provided by way of illustration, and are not intended to limit the present invention.

### DESCRIPTION OF THE FIGURES

FIG. 1. Suppressor effect of the anti-N-PCT antibody of the inhibition of food intake by N-PCT. Suppressor effect of 10 μg of anti-N-PCT in the inhibition of the intake produced by N-PCT measured during 24 hours after a single intracerebroventricular (icy) administration of 5 μg of N-PCT or a carrier (aCSF) in male Wistar rats (n=8/group), at the start of the darkness phase. ***P<0.001 vs. Icy anti-N-PCT or IgG (control) or vs. Animals treated with anti-N-PCT+N-PCT.

FIG. 2. Time-dependent effect of LPS on body temperature (Tb) and production of ACTH, CT and cytokines in plasma. The rats are injected intraperitoneally (ip) with LPS (15 mg/kg) or an equivalent volume of PFS (saline serum free from pyrogens) at the start of the darkness phase. The animals (n=10-12) were sacrificed 1, 3, 6, or 12 hours after LPS administration. ACTH, CT, TNF-α, IL-1β and IL-10 were measured in plasma by ELISA. The columns represent the averages. The bars indicated the SEM. ND, non-detectable. **P<0.01, ***P<0.001 vs. rats treated with PFS at time 0.

FIG. 3. Hypothalamic expression of the Calca-1 gene, and production of PCT after LPS administration. Hypothalamic expression of the Calca-1 gene (A) and production of PCT in plasma and hypothalamus at different moments after LPS administration. The animals were sacrificed 1, 3, 6, 12 and 18 hours after the ip LPS administration (15 mg/kg) or the equivalent carrier. The expression of Calca-1 was analysed by RT-PCR. The PCT concentrations in plasma and hypothalamus are measured by an immunoluminometric assay. The immune response induced by LPS was correlated with an increase in PCT levels in hypothalamus and plasma. The data represent the average±SEM of 8 rats per time. **P<0.01, ***P<0.001 vs. rats treated with PFS at time 0.

FIG. 4. Photomicrographies of cells and fibres immunoreactive to N-PCT.

Photomicrographies showing cells and fibres immunoreactive to N-PCT, of the arcuate nucleus (ARC) and the ventromedial nucleus (VMH) of normal rats (A) and (B) or with treatment with endotoxins (C-G). The animals (n=4 per each time) were sacrificed 1 (C), 3 (D), 6 (E), 12 (F) and 18 (G) hours after the administration of a lethal dose of LPS ip (15 mg/kg). H and I represent cells and fibres immunoreactive to N-PCT in long-term survivors treated with anti-N-PCT antibody 3 or 7 days after LPS administration. The control experiments include mouse IgG serum as negative control, and staining with primary antibody as negative staining control. Immunostaining was not detected in either case. Abbreviations: 3v third ventricle; ME, median eminence; VGL ventral glial lamina or glial limiter (p. anterior brain). Scale bars: A, 100 μm: B-I, 10 μm.

FIG. 5. Immunofluorescence of GFAP and N-PCT. Double immunofluorescence of the glial fibrillary acidic protein (GFAP) in the VHM (A-C) and in the ARC (D-F) of control rats. It shows the co-localization of GFAP and N-PCT in astrocytes of VHM© and ARC (F). These comparative images show astrocytes containing N-PCT with protoplasmic morphology. Scale bars: 50 μm A-C; 10 μm (D-F).

FIG. 6. Delay in death and improvement of long-term survival in rats treated with anti-N-PCT before treatment with LPS.

Delay in the start of death and improvement in the long-term survival of rats treated with anti-N-PCT before the lethal administration of bacterial endotoxin. Groups of 18-20 animals per group were injected ip with PFS, IgG control (100 μg/kg) or anti-N-PCT (100 μg/kg), 1 hour before the high dose of LPS (15 mg/kg, at time 0). The data shown as the accumulated percentage of rats still alive within each interval and the survival after 8, 12, 18, 24 48, 72, and 168 hours. After 48 hours no control rat survived in comparison with 85% of survival of the rats treated with anti-N-PCT (P<0.001). There was no change in survival after 48 hours.

FIG. 7. Delay in death and improvement of long-term survival in rats treated with anti-N-PCT after treatment with LPS.

The animals were treated with anti-N-PCT or IgG after LPS administration (doses similar to the aforementioned 15 mg/kg). In comparison to the previous group, the animals that received anti-N-proCT 1 and 3 h after LPS reached a mortality of 10% after 12 h, 40% after 24 h, and 54% after 48 h. This protection level was maintained throughout the course of the study. The animals (FIG. 7). The animals that survived the first 48 h completely recovered and continued alive for at least another three weeks (time when the monitoring study finished). The single administration of anti-N-PCT to normal rats did not produce toxic effects.

### EXAMPLES

The specific examples provided in this patent document serve to illustrate the nature of the present invention. These examples are only included with illustrative purposes and cannot be interpreted as limitations of the invention claimed here. Therefore, the examples described below illustrate without limiting the field of application thereof.

### Example 1

### Effects of the Intracerebroventricular Administration of Anti-N-PCT in N-PCT-Induced Anorexia

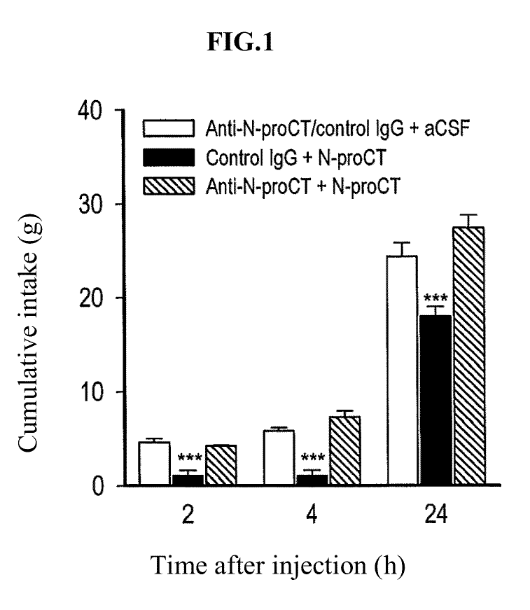

The effect of the cerebroventricular administration in the intake of is shown in FIG. 1. The intake was measured 2, 4 and 24 hours after a single intracerebroventricular injection of N-PCT (5 μg), at the start of night-time feeding. In accordance with previous data, the icy administration of N-PCT reduced food intake during 24 hours. In comparison with the administration of the carrier, N-PCT reduced the intake by 78% during the first 2 hours and 83% during the first 4 hours (P<0.001). The accumulated intake during 24 hours was reduced 26% with respect to the controls. The treatment with anti-NPCT reversed these effects (p<0.001). This confirms the specificity of the antibodies used. Furthermore, these data demonstrate the implication of NPCT in metabolic stress.

### Example 2

### Effects of a Lethal Dose of LPS on the Behaviour and Plasma Levels of ACTH, CT and Cytokines

After the administration of a high dose of LPS (15 mg/kg, ip), all rats showed serious symptoms of disease. They quickly became lethargic and anorexic and showed standing on end of the hair, chromodacryorrhoea and diarrhoea. Many of the animals showed a short hypothermia followed by fever. The animals seemed moribund after 12 hours and none survived 48 hours.

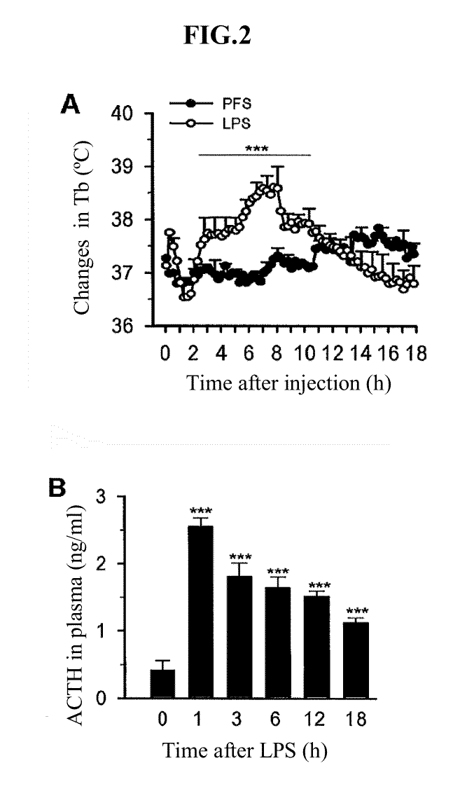

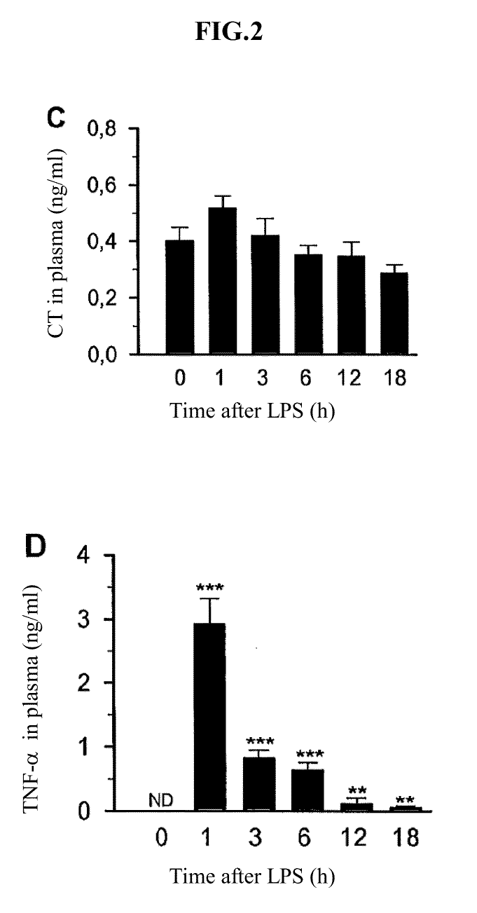

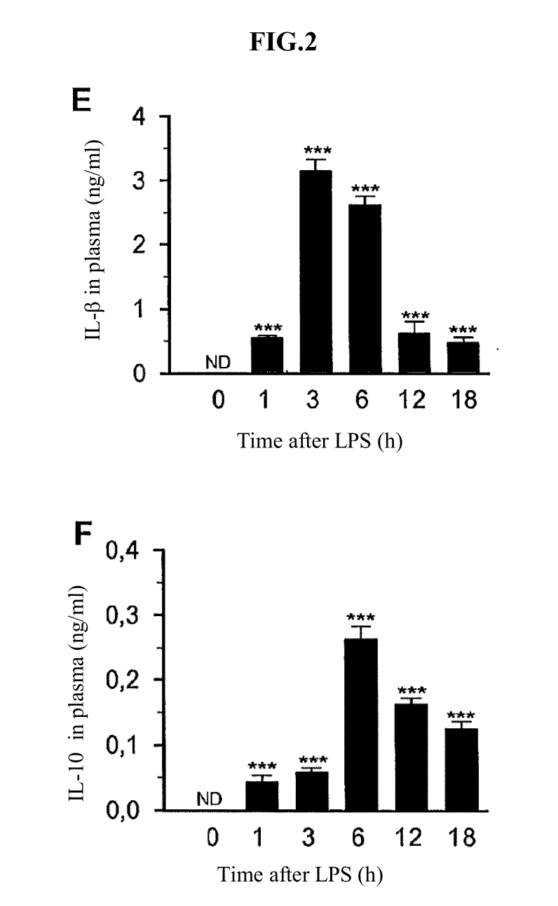

As shown in FIG. 2A, the administration of 15 mg/kg of LPS caused a decrease in body temperature (Tb) from 30 minutes after the administration of LPS, and reached a maximum after one and a half hours. (36.5±0.06° C.). Within the first 2 hours after LPS administration, Tb began to gradually increase, reaching a maximum after 8 hours (38.67±0.15° C.), which lasted 12 hours (37.49±0.12° C.). Tb was not altered by the administration of PFS. The plasma levels of ACTH, CT, TNF-α, IL-1β and IL10 were also measured after the injection of LPS (FIG. 2). As shown in FIG. 2B, LPS increases plasma ACTH levels, reaching 2.5±0.1 ng/ml one hour after administration. The levels then decrease after 3 hours and fall to 1.1±0.1 ng/ml after 24 hours. On the other hand, there is no increase in CT concentration (FIG. 2C). It also analysed the expression of TNF-α, IL-1β and IL-10 in plasma. TNF-α and IL-1β increased reaching a maximum after 1 and 3 hours, respectively. The decrease in TNF-α started to occur after 3 hours. In the case of IL-1β, the levels are maintained until 6 hours and then decrease 12 hours after LPS administration. IL-10 levels increase more gradually and reach a maximum after 6 hours and decrease after 18 hours.

### Example 3

### Expression of the Calca-1 Gene and Production of PCT in Plasma and Hypothalamus After LPS Administration

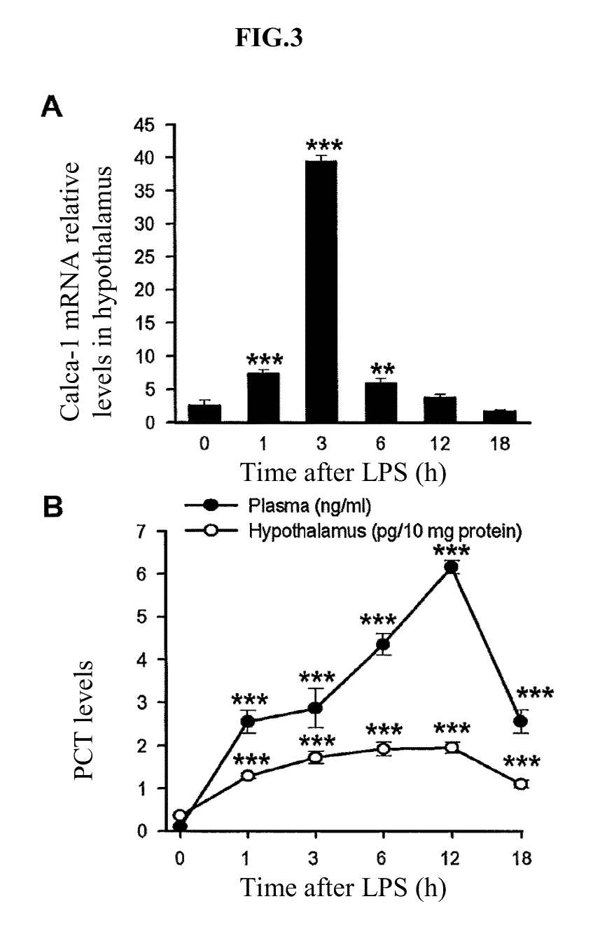

The Calca-1 gene was expressed at high levels in the hypothalamus after LPS administration (FIG. 3A). Similarly to PCT levels, the expression of Calca-1 began its increase one hour after administration. It reaches the maximum after 3 hours and begins to decrease after 6 hours, reaching basal levels after 12 hours. There is a low expression of Calca-1 in the rats treated with PFS.

LPS administration produces a fast and striking increase in PCT levels in plasma and hypothalamus. Between 1 and 3 hours. They reach a maximum after 12 hours and are maintained until the death of the animal. The increases are of 6000 and 540 times after 12 hours in plasma or hypothalamus, respectively. Those increases of PCT in plasma correlate with the production of PCT in the hypothalamus. There appears a fast increase after one hour that is maintained over time.

### Example 4

### Increase in N-PCT in Brain Regions Involved in Behaviour

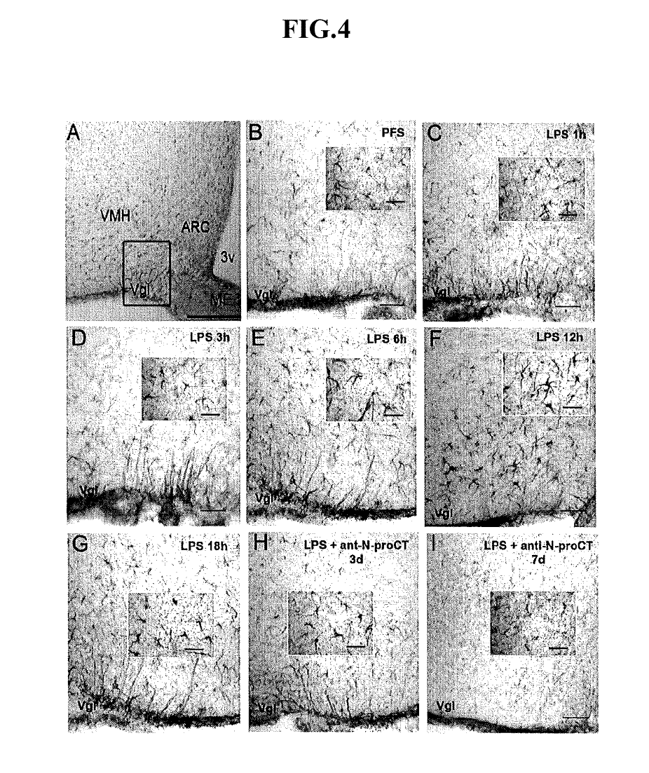

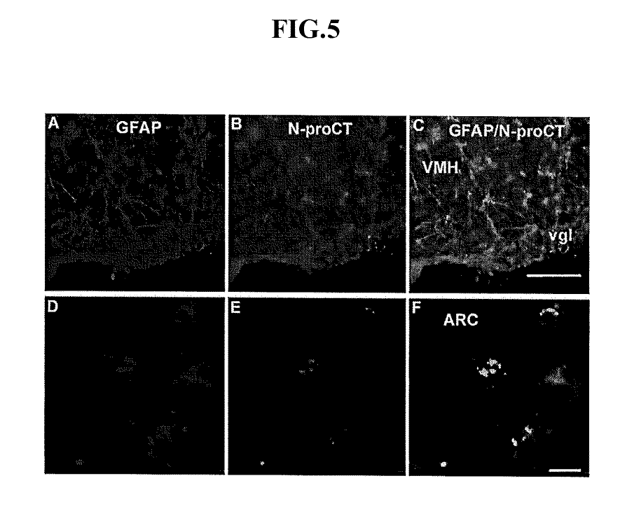

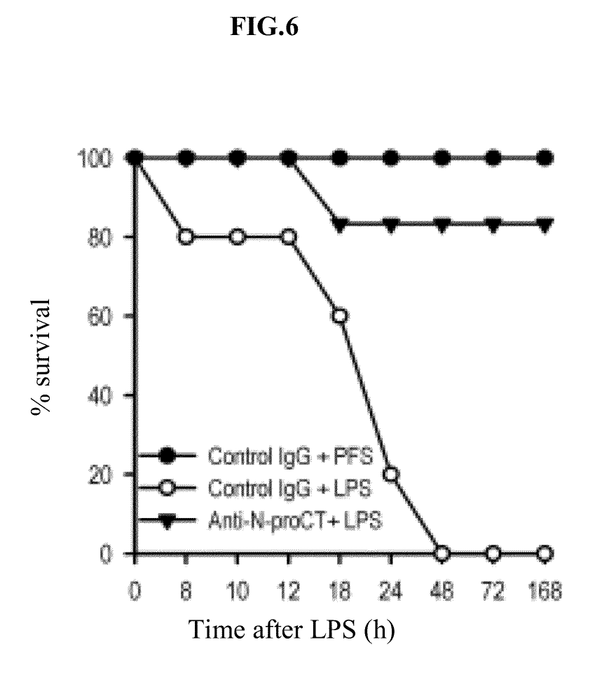

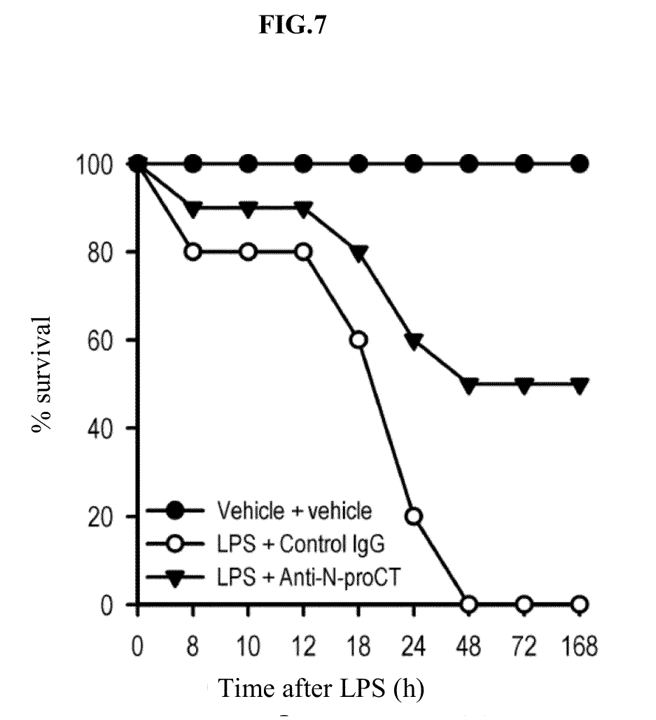

The time-dependent distribution template of N-PCT was studied in the brain after LPS administration. The structures involved in disease such as the hypothalamus were analysed in greater detail. In FIG. 4, the N-PCT immunoreactivity template in normal rats is similar to that present in treated rats. In any form, observations were observed after LPS administration. An intense time-dependent expression of N-PCT is observed in hypothalamic regulatory regions such as VHM, ARC or Vgl (FIG. 4C-G). N-PCT is clearly intracellular and most of the positive N-PCT cells are astrocytes.

A double marking was performed on N-PCT and GFAP for the identification of the astrocyte cell population (FIG. 5). The number of positive cells was increased with treatment with LPS. The immunoreactivity template in normal rats was similar to that found in rats pretreated with anti-NPCT before LPS administration FIG (4H-I). This suggests that the N-PCT may play an important role in the pathways through which the endotoxins induce the immune response.

### Example 5

### Protector Effect of N-PCT Immunoneutralization

On the other hand, the role of endogenous N-PCT was studied in LPS-induced mortality. N-PCT was specifically neutralized with an anti-N-PCT antibody 1 hour before LPS administration. Survival was analysed during 7 days. Death occurred from 8 to 48 hours after LPS administration. As shown in FIG. 6, the passive immunization of rats with a single dose of anti-M-PCT increased the survival rate 100% in comparison with 80% in animals treated with IgG control, from 8 to 12 hours after LPS administration. After 18 hours, the survival was 60% in control rats compared with 85% of rats treated with anti-N-PCT. After 2 days the death in the control rats was 100%, whilst in the rats treated with anti-N-PCT the death was 15% from the first day until the end of the study.

The rats that survived 2 days showed an improvement from that time on, represented by greater mobility and they did not shown lethargy or diarrhoea. 7 days after administration. None of the animals treated with anti-N-PCT showed signs of systemic infection. This suggests that N-PCT has a relevant role in the response that led to survival in the event of septic shock.

### Example 6

### Neutralization of the Circulating N-PCT. It Normalizes the Levels of PCT and Cytokines in Animals with Long-Term Survival

Later, experiments were developed to determine if the circulating N-PCT regulates the balance between the pro and anti-inflammatory cytokines. In animals treated with anti-N-PCT the levels of cytokines TNF-α, IL-1β, IL-10, as well as of PCT returned to basal situation in comparison with the untreated rats. These changes are associated with the normalization of PCT production and immunoreactivity to N-PCT in the hypothalamus. This treatment had no effect on plasma CT levels. Interestingly, the treatment with anti-N-PCT produced an increase in plasma ACTH concentration. All this suggests that N-PCT has a relevant role in the mechanisms whereby LPS induces death, and that the peripheral administration of the anti-N-PCT antibody prevents death reducing the levels of LPS-induced pro-inflammatory cytokines.

TABLE 1
Effect of the immunoneutralization of N-PCT in the levels
of cytokines, ACTH, CT and PCT in plasma 3 and 7 days
after LPS administration, in surviving animals.

| | Time after LPS | Anti-N-PCT + | Anti-N-PCT + |
|---|---|---|---|
| Mediator | administration | PFS | LPS |
| TNF-□ | 3 d | ND | ND |
| | 7 d | ND | ND |
| IL-1□ | 3 d | ND | 0.08 ± 0.02 |
| | 7 d | ND | ND |
| IL-10 | 3 d | ND | ND |
| | 7 d | ND | ND |
| ACTH | 3 d | 0.57 ± 0.12 | 1.68 ± 0.45 |
| | 7 d | 0.48 ± 0.10 | 0.75 ± 0.23 |
| CT | 3 d | 0.39 ± 0.10 | 0.46 ± 0.06 |
| | 7 d | 0.43 ± 0.08 | 0.41 ± 0.12 |
| PCT | 3 d | 0.09 ± 0.01 | ND |
| | 7 d | 0.09 ± 0.01 | ND |

The animals were treated intraperitoneally with 200 μg/kg of control serum or anti-N-PCT serum 1 h before LPS administration (15 mg/kg).
The data are the mean ± standard error of 7 rats per group. ND: not detectable. The IgG + LPS group has not been included as no animal survived 3 days.

### Materials and Methods Used

### Materials

To perform the experiments, synthetic N-PCT was used of sequence SEQ ID NO:5 identical to human N-PCT, obtained from Bachem (Bubendorf, Switzerland)

Functional Antagonism of N-PCT with Specific Antibodies, in Vivo.

Before investigating the therapeutic potential of the anti-N-PCT antibodies in the hyperproduction of cytokines and LPS-induced death, the specificity of the antibodies was analysed in N-PCT-induced anorexia. The rats were anaesthetized intraperitoneally (ip) with a mixture of ketamine (100 mg/kg) and xylazine (5 mg/kg), and a cannula was placed in the lateral ventricle (Tavares et al. 2007 Endocrinology 148:1891-1901). The efficiency of the cannula was tested one week after its placement by the elicitation of the intake of liquid by angiotensin II (Johnson et al. 1975 Brain Res 86:399-418). Only animals with the cannula correctly positioned were used.

After recovery from surgery, the animals got into the habit of handling by the faked injection through the cannula, during at least three days of the experimentation to reduce stress.

The day of the administration, a stainless steel injector was introduced through the cannula and to groups of animals with similar weights (n=8 for each treatment), they were administered intracerebroventricularly (icy) with anti-N-PCT antibodies (10 μg/5 μl) or an equivalent dose of rabbit IgG 15 minutes before administering 5 μg per rat of N-PCT or an equivalent volume of aCSF (5 μl). The compounds were dissolved in aCSF before each experiment and they were injected by gravity through a polyethylene tube connected to an injector cannula. To avoid the reflux, the catheter was sealed. After administration, each animal was given a weighed quantity of food. To analyse the intake, the food not indigested was weighed 2, 4 and 24 hours after treatment. No differences were observed between the control rats and those treated with anti-N-PCT before administration of N-PCT.

### LPS-Induced Systemic Immune Responses

Before the induction of systemic inflammation, each animal was anaesthetized as in example 1. A radiotransmitter with a temperature sensor (model PDT-4000; Mini-Mitter, Sunriver, Oreg.) was implanted in the peritoneum. The body temperature was monitored constantly using the VitalView system (Mini-Mitter) (Tavares et al. 2005). All temperature measurements were taken at 5 minute intervals in conscious animals and they were represented as the average of each hour. At the end of experimentation, the rats were weighed and randomly distributed in groups. The experiments were performed 1 week after implantation of the transmitter.

To see the systemic response, each rat received a high dose of LPS (15 mg/kg, ip) at the start of the darkness phase. It is known that this dose produces a strong inflammatory and immunostimulating response in the periphery and in the brain similar to the changes appearing in thermal shock in humans (Givalois et al. 1994). The control rats received an injection of an equivalent volume of PFS (pyrogen-free saline serum). The animals were sacrificed 1, 3, 6, 12 and 18 hours after administration by decapitation to avoid the effects of the anaesthesia in the changes in the LPS-induced cytokine levels. Blood samples were taken from the trunk in plastic tubes containing heparinized saline serum (50 U/ml) and a protease inhibitor. The samples are centrifuged (3000 g, 10 min, 4° C.) and the plasma was stored at −80° C. until the immunochemical analysis. During the autopsy, the brain is obtained and the hypothalamus is dissected with a scalpel. The hypothalamic block is obtained by rostral cuts transversal to the optic chiasm, and caudal to the mamillary bodies. Lateral cuts were made in the lateral sulcus of the hypothalamus. The complete hypothalamus was frozen in liquid nitrogen and stored at −80° C. until its analysis by RT-PCR and extraction and quantification of PCT.

### Expression of the Calca-1 Gene in Hypothalamus Samples

The expression of the CT transcript after LPS administration was performed on hypothalamus samples by RT-PCR after 1, 3, 6, 12, and 18 (n=10-12 rats per point). The brain block containing the hypothalamus was quickly dissected in dry ice and frozen by immersion in liquid nitrogen to preserve the integrity of the RNA. The frozen tissue was disintegrated in a mortar submerged in liquid nitrogen. The total RNA was obtained using TriPure Isolation Reagent® (Roche applied Science, Indianapolis, USA) in accordance with the manufacturer's instructions. The RNA was quantified by Quant-It RiboGreen RNA Reagent (Molecular Probes). cDNA was synthesized by AffinittyScript QPCR cDNA Synthesis Kit (Stratagene, La Jolla, Calif., USA). The primers for the RT-PCR were designed using the software Beacon Designer Software (Premier Biosoft, Palo Alto, Calif.), in accordance with the sequence of the Calca gene of the NM_{—}017338 rat (National Center for Biotechnology Information Database). The primers used for RT-PCR were the primers of sequence SEQ ID NO:3 and SEQ ID NO:4 (Eurogentec S. A., Liege, B E). In parallel, the expression of mRNA of the microglobulin-β2 was analysed using Mouse Endogenous Control Panel Kit (TATAA Biocenter AB, Goteborg, Sweden), as internal control for normalization. The cDNA samples were denatured during 10 minutes at 95° C. and they were then amplified using 40 30-second cycles at 95° C., one minute at 60° C., 30 seconds at 72° C., and for the dissociation curve, 1 minute at 95° C., 30 seconds at 60° C. and 30 seconds at 95° C. Each sample was amplified using 25 ml of Brilliant SYBR Green QPCR Master Mix (Mx3005P instruments) and 0.15 mM of each oligonucleotide in a Mx3005P Real-Time PCR (Stratagene, La Jolla, Calif., USA) system. The level of each specific cDNA was analysed in the exponential phase and was normalized with the expression level of microglobulin-β2 of each sample. Each cDNA sample was measured in triplicate.

### Quantification of PCT in Plasma and Hypothalamus.

PCT was quantified by the LUMItest® proCT method (Brahms Diagnostica; Germany) by the previously described procedure (Ojeda et al. 2006. Neurosci Lett. 408: 40-45; Tavares et al. 2007. Endocrinology. 148: 1891-1901). The hypothalamus samples (average weight 40 mg), were submerged in HCl 1Normal, they were homogenized and centrifuged (10000 g during 30 minutes at 4° C. The recovery after extraction was analysed in the hypothalamic samples 1, 3, 6, 12 and 18 hours after LPS administration (n=8 rats per point), and the data were corrected based on the protein concentration (Lowry method; Bio-Rad laboratories). The supernatant of the hypothalamic samples and the plasma from the blood samples were analysed in duplicate. Performing various freezing-defrosting cycles of the samples and reagents used was avoided. The PCT test in blood was adjusted to the rat's blood and is equivalent to human blood (coefficient of variation less than 10%). This immunoluminetric assay has a detection limit of 0.1 ng/ml. The intra- and inter-assay variations were less than 8%.

### Quantification of Cytokines, ACTH and CT in Plasma.

The cytokines and CT levels in plasma were determined by commercial ELISA kits for TNF-α (Pierce Biotechnology Inc., IL, USA), IL-1β (R&D Systems Europe, Abingdon, UK), IL-10 (ILB, Hamburg, Germany), and CT (Phoenix Pharmaceutical Inc., Belmont, Calif.). The plasma cytokines were measured according to the method described above (Tavares et al. 2005. Clin Diagn Lab Immunol 12:1085-1093). The detection limit was 15 pg/ml for TNF-α, 5 pg/ml for 1L-1β, 10 pg/ml for IL-10 and 0.36 ng/ml for CT. The coefficients of variation between tests were less than 5%. Adrenocorticotropic hormone (ACTH) was measured in plasma by immunoenzymatic assay using a ACTH EIA commercial kit (Phoenix Pharmaceuticals, Belmont, Calif., USA). ACTH was extracted from the plasma using a buffer containing 1% triacetofluoretic acid and by the C-18 column technique. The samples extracted were dehydrated using the Eppendorf Vacufuge, and the resulting samples were stored at −80° C. The samples were reconstituted 24 hours before the experiments in the test buffer. The detection range of the kit was 0-25 ng. All plasma samples were analysed in duplicate.

### LPS-Induced Expression of N-PCT in the Hypothalamus

To see the activation of N-PCT in the hypothalamus, the immunoreactive N-PCT was analysed in the hypothalamus at different times after LPS administration.

### Treatment and Sample Taking

Different groups of rats were injected intraperitoneally with LPS (15 mg/kg) or an equivalent volume of PFS at the start of the darkness phase. The animals were strongly anaesthetized 1, 3, 6, 12 and 18 hours after LPS administration (n=4 rats per point) and they were perfused through the aorta with 50 ml of 0.1M cold phosphate buffered saline (SPB) (pH 7.4) followed by 300 ml of 0.1M PBS with 4% paraformaldehyde. The brains were obtained and they were fixed all night with the same fixing solution. They were passed to a cryoprotectant solution (30% sucrose in 0.1 M PBS at 4° C. until sinking. The fixed brains were mounted with Tissue Tek (Raymond Lamb, London, UK) and frozen in dry ice. 30 μm cuts were made using a cryostat (Leica Microsystems, SA, Barcelona) through the rostrocaudal extension of the hypothalamus, beginning in a position 200 μm after the bregma.

### Immunohistochemistry

A polyclonal rabbit antibody generated against a highly conserved segment of human N-PCT (AbD Serotec) was used to identify the NPCT. The cuts were quickly washed in PBS and are incubated during 15 minutes in a solution with 6% H₂O₂. After washing them with PBS, they were incubated with a blocking solution (5% goat serum and 0.1% saponin in PBS) for 30 minutes at ambient temperature, and they are then incubated for 24 hours at 4° C. with anti-N-PCT antibody (dilution 1:200). The cuts were then incubated with a biotinylated anti-goat IgG antibody (1:200, biogenex) and with the avidin horse radish peroxidase complex (Vectastain Elite ABC kit, Vector). Finally, the cuts are incubated with diaminobenzidine (Dako, Carpinteria, Calif.). As soon as they are stained brown, they are washed, counterstained with blue violet, dehydrated and covered. Some cuts are analysed without counterstaining. The brain sections were post-fixed in glass slides under the same conditions, to avoid the possible of variations in N-PCT staining. All antibodies were diluted in PBS with 0.1% saponin.

For the simultaneous detection of N-PCT with GFAP a double immunofluoresence was performed in the hypothalamus cuts. The cuts were incubated for 3 days at 4° C. with the first primary antibody (anti-N-PCT 1:200). The cuts were later incubated sequentially with a secondary antibody marked with anti-rabbit FITC (fluorescein; Vector, 1:500, q hour), rabbit serum in PBS (4% during 1 hour), anti-rabbit antibody (1:500 during one hour, Jackson ImmunoResearch), and a rabbit anti-GFAP polyclonal antibody at a dilution 1:4000 (Dako) throughout the night. Finally, an anti-rabbit antibody conjugated with TRICT (rhodamine, Dako, dilution 1:100) during 1 hour. Finally, the cuts are washed and mounted with fluorescent mounting medium (Dako). The cuts are analysed by a fluorescence microscope. The images are processed by Adobe photoshop for adjustment, contract and brilliance.

### LPS-Induced Death.

To analyse the role of the circulating N-PCT in LPS-induced death, different groups of rats (18-20 rats per group) were injected intraperitoneally with 100 μg/kg of anti-N-PCT or with IgG 1 hour before LPS administration (15 mg/kg) at the start of the darkness phase. Survival was studied until 7 days after the injections. The condition of the rats was constantly monitored during the first 8 hours and from that time when considered necessary at minimum intervals of 6 hours, where diverse parameters are analysed such as the movement or gastrointestinal alterations. The animals that appeared moribund or did not respond to external stimuli were sacrificed to avoid their suffering. All experiments were performed by blind analysis.

Different groups of rats were sacrificed by decapitation 3 and 7 days after the injections (n=7 rats per group). Blood from the trunk was collected to analyse cytokines in plasma, PCT, ACTH and CT.

### Generation of Polyclonal Antibodies

For the generation of the polyclonal antibodies a standard animal immunization protocol was carried out. A rabbit serum reactive against N-PCT was obtained. The protocol is carried out by the company Genscript. The immunization process comprises the following steps:
1) Generation of the peptides of sequence SEQ ID NO:1 and/or SEQ ID NO:2
2) Addition of a cysteine at one end of the peptide.
3) Animal immunization
4) Obtainment of antiserum from the immunized animals
5) Purification of the antibodies by a protein A column
6) Performance of an ELISA to analyse the antibodies extracted.

### Generation of Monoclonal Antibodies

For the generation of the monoclonal antibodies of the present invention, a standard immunization process was carried out and a later hybridization and selection of clones. The process comprises the following steps:
7) Generation of the peptides of sequence SEQ ID NO:1 and/or SEQ ID NO:2
8) Animal immunization
9) Obtainment of blood from said analysis and ELISA analysis
10) Selection of the animals with best production and obtainment of splenocytes
11) Fusion of splenocytes with immortalized cells to obtain hybridomas
12) Selection of the hybridomas with best production
13) Obtainment of the antibodies.

1. An isolated nucleotide sequence which codes for amino acid sequence SEQ ID NO:1, for amino acid sequence SEQ ID NO: 2, or for a biologically active fragment or variant thereof.

2. A peptide that consists of the amino acid sequence SEQ ID NO:1, or a biologically active fragment or variant of SEQ ID NO:1 or a peptide that consists of the amino acid sequence SEQ ID NO: 2, or a biologically active fragment or variant of SEQ ID NO: 2.

3. A genetic construction comprising:
a) the isolated nucleotide sequence of claim 1, or
b) a nucleotide sequence according to (a) included in an expression vector, operationally bound to, at least, one promoter.

4. A host cell comprising the isolated nucleotide sequence of claim 1.

5. A host cell comprising the genetic construction of claim 3.

6. A method for the generation of antibodies comprising the use of a peptide that consists of the amino acid sequence SEQ ID NO:1, or a biologically active fragment or variant of SEQ ID NO:1 or a peptide that consists of the amino acid sequence SEQ ID NO: 2, or a biologically active fragment or variant of SEQ ID NO: 2.

7. The method for the generation of antibodies according to claim 6 comprising:
a) adding a cysteine at one of the ends of said peptide,
b) conjugating it with KLH,
c) immunizing a mammal animal with a peptide according to (a),
d) extracting the antiserum from the animal, and
e) purifying the antibody that specifically recognizes N-procalcitonin.

8. The method for the generation of antibodies according to claim 6, comprising:
a) adding a cysteine at one of the ends of said peptide,
b) conjugating it with KLH,
c) immunizing a mammal animal with a peptide according to (a),
d) analysing the titration against the peptide of step (a) by ELISA, in the mammal animal of step (c),
e) fusing the splenocytes of the host animals for the generation of immortalized cell lines,
f) expanding the clones, and
g) selecting the best producers.

9. An antibody generated by a method comprising:
a) adding a cysteine at one of the ends of a peptide of claim 2,
b) conjugating it with KLH,
c) immunizing a mammal animal with a peptide according to (a),
d) extracting the antiserum from the animal, and
e) purifying the antibody that specifically recognizes N-procalcitonin or by a method comprising:
a) adding a cysteine at one of the ends of a peptide of claim 2,
b) conjugating them with KLH,
c) immunizing a mammal animal with a peptide according to (a),
d) analysing the titration against the peptide of step (a) by ELISA, in the mammal animal of step (c),
e) fusing the splenocytes of the host animals for the generation of immortalized cell lines,
f) expanding the clones, and
g) selecting the best producers.

10. The antibody according to claim 9 that specifically recognizes N-procalcitonin.

11. The antibody according to claim 9, capable of binding to a peptide containing the amino acid sequences SEQ ID NO:1 or SEQ ID NO:2.

12. A method of treatment in a subject for diseases that develop with alterations of the inflammatory response or for diseases that develop with metabolic stress comprising the administration to said subject of an antibody generated by a method comprising:
a) adding a cysteine at one of the ends of a peptide of claim 2,
b) conjugating it with KLH,
c) immunizing a mammal animal with a peptide according to (a),
d) extracting the antiserum from the animal, and
e) purifying the antibody that specifically recognizes N-procalcitonin. or by a method comprising:
a) adding a cysteine at one of the ends of a peptide of claim 2,
b) conjugating them with KLH,
c) immunizing a mammal animal with a peptide according to (a),
d) analysing the titration against the peptide of step (a) by ELISA, in the mammal animal of step (c),
e) fusing the splenocytes of the host animals for the generation of immortalized cell lines,
f) expanding the clones, and
g) selecting the best producers.

13. The method according to claim 12 where the disease that develops with alteration of the inflammatory response is selected from the list comprising: sepsis, septic shock, cardiogenic shock, post-surgical complications, peritonitis, transplants, autoimmune diseases, obesity, diabetes, bacterial meningitis, neoplasias or neurodegenerative diseases that develop with inflammation.

14. A method for the quantification of N-procalcitonin in serum, cerebrospinal fluid, cell or tissue homogenates comprising the use of an antibody generated by a method comprising:
a) adding a cysteine at one of the ends of a peptide of claim 2,
b) conjugating it with KLH,
c) immunizing a mammal animal with a peptide according to (a),
d) extracting the antiserum from the animal, and
e) purifying the antibody that specifically recognizes N-procalcitonin, or by a method comprising:
a) adding a cysteine at one of the ends of a peptide of claim 2,
b) conjugating them with KLH,
c) immunizing a mammal animal with a peptide according to (a),
d) analysing the titration against the peptide of step (a) by ELISA, in the mammal animal of step (c),
e) fusing the splenocytes of the host animals for the generation of immortalized cell lines,
f) expanding the clones, and
g) selecting the best producers.

15. A diagnostic method of diseases that develop with alterations of the inflammatory response or diseases that develop with metabolic stress, comprising:
(i) obtaining an isolated biological sample from an individual,
(ii) detecting the quantity of N-procalcitonin in the biological sample of (i), by an antibody generated by a method comprising:
a) adding a cysteine at one of the ends of a peptide of claim 2,
b) conjugating it with KLH,
c) immunizing a mammal animal with a peptide according to (a),
d) extracting the antiserum from the animal, and
e) purifying the antibody that specifically recognizes N-procalcitonin, or by a method comprising:
a) adding a cysteine at one of the ends of a peptide of claim 2,
b) conjugating them with KLH,
c) immunizing a mammal animal with a peptide according to (a),
d) analysing the titration against the peptide of step (a) by ELISA, in the mammal animal of step (c),
e) fusing the splenocytes of the host animals for the generation of immortalized cell lines,
f) expanding the clones, and
g) selecting the best producers, and
(iii) comparing the quantities obtained in step (ii) with a reference quantity.

16. The method according to claim 15 which further includes assigning to an individual according to step (i) to the group of individuals with a disease that develops with alteration of the inflammatory response or a disease that develops with metabolic stress, when a quantity of N-procalcitonin is detected in step (ii) greater and statistically significant in comparison with a reference quantity.

17. The method according to claim 15, wherein the disease that develops with alterations of the inflammatory response is selected from the list comprising: sepsis, septic shock, cardiogenic shock, post-surgical complications, peritonitis, transplants, autoimmune diseases, obesity, diabetes, bacterial meningitis, neoplasias or neurodegenerative diseases that develop with inflammation.

18. A diagnostic kit for diseases that develop with alterations of the inflammatory response or for diseases that develop with metabolic stress comprising an antibody generated by a method comprising:
a) adding a cysteine at one of the ends of a peptide of claim 2,
b) conjugating it with KLH,
c) immunizing a mammal animal with a peptide according to (a),
d) extracting the antiserum from the animal, and
e) purifying the antibody that specifically recognizes N-procalcitonin, or by a method comprising:
a) adding a cysteine at one of the ends of a peptide of claim 2,
b) conjugating them with KLH,
c) immunizing a mammal animal with a peptide according to (a),
d) analysing the titration against the peptide of step (a) by ELISA, in the mammal animal of step (c),
e) fusing the splenocytes of the host animals for the generation of immortalized cell lines,
f) expanding the clones, and
g) selecting the best producers.

19. The diagnostic kit according to claim 18, wherein the disease that develops with alterations of the inflammatory response is selected from the list comprising: sepsis, septic shock, cardiogenic shock, post-surgical complications, peritonitis, transplants, autoimmune diseases, obesity, diabetes, bacterial meningitis, neoplasias or neurodegenerative diseases that develop with inflammation.
